(19) United States
(12) Patent Application Publication
(10) Pub. No.: US 2009/0270936 A1
(43) Pub. Date: Oct. 29, 2009

(54) IMPLANTABLE MEDICAL DEVICE WITH COORDINATED VENTRICULAR OVERDRIVE AND TRIGGER MODE PACING

(75) Inventors: Michael E. Benser, Valencia, CA (US); Euljoon Park, Valencia, CA (US)

Correspondence Address: PACESETTER, INC., 15900 VALLEY VIEW COURT, SYLMAR, CA, 91392-9221, US

(73) Assignee: PACESETTER, INC., Sylmar, CA (US)

(21) Appl. No.: 12/111,876

(22) Filed: Apr. 29, 2008

(51) Int. Cl. A61N 1/36

(52) U.S. Cl. 607/15

(57) ABSTRACT

A method and system are provided for providing coordinated ventricular overdrive and triggered pacing through an implantable system. A lead senses signals from a heart to obtain sensed signals representative of tachycardia occurring in at least one chamber of the heart. The lead includes an electrode. A control module detects tachycardia in at least one chamber of the heart and based thereon, initiates an overdrive pacing mode and a triggered pacing mode. The control module controls delivery of overdrive pacing pulses through the electrode to a first chamber of the heart in accordance with the overdrive pacing mode. The control module controls delivery of a triggered pacing pulse through the electrode to the first chamber of the heart in accordance with the triggered pacing mode. The triggered pacing pulse is temporally interspersed with the overdrive pacing pulses. The triggered pacing pulse may be delivered at a time that is independent of, and unrelated to, the timing of the overdrive pacing pulses.

### FIELD OF THE INVENTION

The present invention relates generally to the field of implantable cardiac stimulation. Embodiments of the present invention more particularly relate to methods and systems for improving ventricular function during atrial tachycardia.

### BACKGROUND OF THE INVENTION

The heart can be modeled by a series of pumps that are controlled by an electrical system that regulates the heart rate. An electrical signal originates at the sino-atrial (SA) node near the top of the right atrium. The electrical signal continues in a downward fashion through the “atrio-ventricular” or AV node, to the bundle of His, which branches in the lower chambers of the heart. In normal sinus rhythm, the ventricles contract almost simultaneously. With normal conduction, the cardiac contractions are organized and timed so that the top chambers (the atria) contract before the lower chambers (the ventricles).

Abnormally fast heart rates are called tachycardias. As used herein, the term tachycardia means a heartbeat at a rate which is abnormally high, or any arrhythmia involving recognizable heartbeat patterns containing repetitions which are in excess of a desired heartbeat range. When the ventricular chambers beat too quickly, the arrhythmia (i.e., unusual heart rhythm) is known as ventricular tachycardia (VT).

Under certain circumstances, a pacemaker, implantable cardioverter defibrillator, and the like may compensate for abnormal operation of a heart by pacing (e.g., stimulating) one or more of the atria and/or ventricles. To stimulate the heart, a typical pacemaker generates a series of electrical signals which are applied to the heart via one or more electrodes implanted in the heart (e.g., proximate to ventricular and/or atrial chambers).

Modern programmable pacemakers are generally of two types: (1) single chamber pacemakers, and (2) dual-chamber pacemakers. In a single chamber pacemaker, the pacemaker provides stimulation pulses to, and senses cardiac activity within, a single-chamber of the heart (e.g., either the right ventricle or the right atrium). In a dual-chamber pacemaker, the pacemaker provides stimulation pulses to, and senses cardiac activity within, two chambers of the heart (traditionally both the right atrium and the right ventricle). More recently, certain pacemakers, called bi-ventricular (BiV) pacemakers, have been developed to independently stimulate the right and left ventricles, with separate leads. These bi-ventricular pacemakers provide for a means of synchronizing stimulation of the left and right ventricles in patients who otherwise would exhibit dyssynchronous, and therefore inefficient, ventricular contraction. Bi-ventricular pacemakers operate in numerous modes, such as atrial tracking pacing mode, non-atrial tracking pacing mode, ventricular triggered mode, and the like.

In the atrial tracking (DDD) bi-ventricular pacing mode, pacing or sensing occurs in the atria, then (after a programmed time period) pacing occurs in the ventricles. In this manner, the bi-ventricular pacing tracks the rate of the atria (where the heart beat starts). Unfortunately, in some instances, a given patient may develop fast atrial rhythms which result from a pathologic arrhythmia such as a pathologic tachycardia, fibrillation or flutter.

Standard modern dual-chamber pacemakers now prevent undesirable tracking of certain atrial arrhythmias by automatically switching the pacemaker's mode of operation from the atrial tracking pacing mode to the non-atrial tracking pacing mode. For example, the pacemaker may temporarily switch from the atrial tracking pacing mode (DDD) to the non-atrial tracking pacing mode (DDI or VVI) for a fixed number of stimulation pulses if the sensed atrial activity indicates an atrial arrhythmia is present. This functionality has carried over to bi-ventricular devices.

During atrial fibrillation (AF), a standard modern dual-chamber bradycardia pacemaker switches to a non-atrial tracking mode to prevent rapid, irregular ventricular pacing. When the intrinsic ventricular response to the AF is faster than the pacing rate, pacing is inhibited. This is an appropriate response for a standard demand type bradycardia pacemaker, which is designed to prevent the patient's heart rate from falling below a certain minimum limit. For heart failure (HF) patients with bi-ventricular devices, the benefit of the device is best realized with continuous, or nearly continuous, bi-ventricular pacing. Thus, during rapidly conducted AF, the patient's device may be functioning suboptimal.

More specifically, when a patient goes into AF, the typical response of a BiV pacemaker (also referred to herein simply as “the device”) is to switch from an atrial tracking mode to a non-atrial tracking mode to prevent the fast, irregular tracking of the atrial fibrillation by the device. When the device mode switches, it typically goes to either a fixed BiV pacing rate, or to an adjustable BiV pacing rate that provides rate response based on the patient's level of activity or other available indicators of the patient's physiologic need. For the sake of consistency, the BiV pacing rate during mode switch (i.e., during a non-atrial tracking bi-ventricular pacing mode) will be hereafter referred to as the “mode switch base rate” or simply as “MSBR”. If the patient's intrinsic rate is above the MSBR, the pacemaker's output is inhibited.

One form of biventricular pacing is referred to as cardiac resynchronization therapy (CRT). Many patients, with CRT devices, have intact atrio-ventricular (A-V) conduction but experience paroxysmal AF (PAF) (e.g., recurrent AF episodes that terminate spontaneously). Patents with a CRT device and with PAF may not warrant ablation of the AV node. In these patients, when the AF occurs, the CRT device typically switches to a VVI pacing mode with the MSBR. It may be advantageous for the CRT device to be programmed with an elevated MSBR, for example 70-80 bpm depending on the patent's intrinsic breakthrough rate during AF. However, today only a small minority of patients actually have their CRT device programmed with an elevated MSBR. In the majority of patients, the MSBR setting is maintained at its default setting (e.g. 60 bpm). Hence, in the majority of patients who experience AF, when the CRT device switches to the VVI pacing mode, with the default MSBR setting, patients experience a significantly higher net ventricular rate. Thus, the great majority of beats are intrinsic and not biventricularly paced. The patient may experience significantly higher ventricular rate irregularity.

Moreover, in CRT patients, the LV hemodynamics of intrinsic beats may be inferior to that of paced beats.

A need remains for more effective techniques and devices for treating patients with atrial tachyarrhythmia who also have some level of disorder in the ventricular activation sequence associated with intrinsic conduction.

### BRIEF SUMMARY OF THE INVENTION

In accordance with an embodiment, an implantable system for providing coordinated ventricular overdrive and triggered pacing comprises at least one lead and a control module. The lead senses signals from a heart to obtain sensed signals representative of tachycardia occurring in at least one chamber of the heart. The lead(s) include at least one electrode. A control module detects tachycardia in at least one chamber of the heart and based thereon, initiates an overdrive pacing mode and a triggered pacing mode. The control module controls delivery of overdrive pacing pulses through the electrode to a first chamber of the heart in accordance with the overdrive pacing mode. The control module controls delivery of a triggered pacing pulse through the electrode to the first chamber of the heart in accordance with the triggered pacing mode. The triggered pacing pulse is temporally interspersed with the overdrive pacing pulses.

In accordance with at least one embodiment, a method of pacing ventricles during tachycardia comprises sensing atrial tachycardia and delivering overdrive pacing pulses to at least one of left and right ventricles at an overdrive pacing rate. An intrinsic event associated with at least one of the left and right ventricles is sensed and a triggered pacing pulse is delivered to at least one of the left and right ventricles based on the intrinsic event.

In accordance with yet another embodiment, an implantable stimulation system comprises a first lead, a second lead and an implantable stimulation device. The first lead senses signals from a heart to obtain sensed signals representative of at least one of an intrinsic event and tachycardia occurring in at least one chamber of the heart. The first lead includes at least a first electrode for delivery of a pulse to a first chamber of the heart. The second lead senses signals from the heart to obtain sensed signals representative of an intrinsic event. The second lead includes at least a second electrode for delivery of a pulse to a second chamber of the heart. The implantable stimulation device is in communication with the first and second leads and comprises a control module configured to detect tachycardia in at least one chamber of the heart and based thereon, initiates an overdrive pacing mode and a triggered pacing mode. The control module is configured to deliver overdrive pacing pulses through at least one of the first and second electrodes in accordance with the overdrive pacing mode, and to deliver triggered pacing pulses through at least one of the first and second electrodes upon detection of the intrinsic event.

### BRIEF DESCRIPTION OF THE DRAWINGS

FIG. 1 illustrates a simplified diagram of an implantable stimulation system for delivering stimulation to areas of the heart with an implantable stimulation device according to an embodiment of the present invention.

FIG. 2 illustrates a functional block diagram of the implantable stimulation device of FIG. 1 according to an embodiment of the present invention.

FIG. 3 illustrates a flowchart implementing a method for providing coordinated ventricular overdrive pacing and triggered pacing using the stimulation device of FIG. 1 according to an embodiment of the present invention.

FIG. 4 illustrates a method for resynchronizing the overdrive timing based on a trigger paced event in accordance with an embodiment of the present invention.

FIG. 5 illustrates a block diagram of exemplary manners in which embodiments of the present invention may be stored, distributed and installed on computer readable medium.

### DETAILED DESCRIPTION OF THE INVENTION

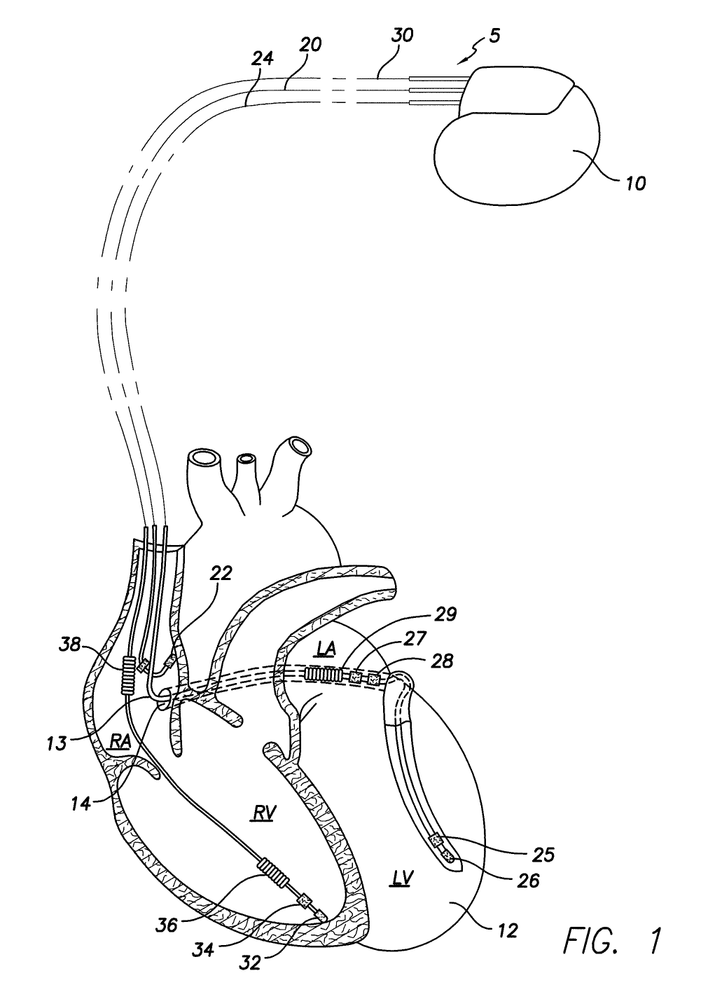

FIG. 1 illustrates a simplified diagram of an implantable stimulation system 5 having an implantable medical stimulation device 10 in electrical communication with one or more leads, such as leads 20, 24 and 30 implanted in or proximate a patient's heart 12 for delivering multi-chamber stimulation (e.g. pacing, high voltage shocks and the like). The device 10 is programmable, by an operator, to set certain operating parameters, as well as therapy-related parameters. The device 10 may be configured to operate with various configurations of leads, one of which is shown in FIG. 1.

To sense atrial cardiac signals and to provide right atrial chamber stimulation therapy, the stimulation device 10 is coupled to an implantable right atrial lead 20 having at least an atrial tip electrode 22, which typically is implanted in the patient's right atrial appendage or septum. An optional atrial ring electrode 23 may also be coupled to the right atrial lead. Stimulation device 10 may be a pacing device, a pacing apparatus, a cardiac rhythm management device, an implantable cardiac stimulation device, an implantable cardioverter/defibrillator (ICD) and/or a cardiac resynchronization therapy (CRT) device.

To sense left atrial and ventricular cardiac signals and to provide left chamber pacing therapy, the stimulation device 10 is coupled to a “coronary sinus” lead 24 that may be placed in the “coronary sinus region”. The coronary sinus lead 24 may receive atrial and ventricular cardiac signals and deliver left ventricular pacing therapy using at least one of a left ventricular (LV) tip electrode 26 and a LV ring electrode 25. Left atrial pacing therapy uses, for example, first and second left atrial (LA) ring electrodes 27 and 28 connected to the coronary sinus (CS) lead 24. Coronary sinus lead 24 can also include a pair of right atrial (RA) ring electrodes 13 and 14 that may be used to provide right atrial chamber pacing therapy.

A right ventricular lead 30 includes at least one of an RV tip electrode 32, an RV ring electrode 34, an RV coil electrode 36, and a superior vena cava (SVC) coil electrode 38 (also known as a right atrial (RA) coil electrode). The right ventricular lead 30 is capable of receiving cardiac signals, and delivering stimulation in the form of pacing and shock therapy to the right ventricle.

The stimulation device 10 operates in various modes, as explained below in more detail. By way of example only, when the stimulation device 10 operates in a triggered pacing mode, the coronary sinus lead 24 and right ventricular lead 30 sense activity in the left and right ventricles, respectively. When an intrinsic event is detected in one ventricle, the coronary sinus lead 24 and/or right ventricular lead 30 deliver ventricular pacing therapy in the other ventricle or in both ventricles.

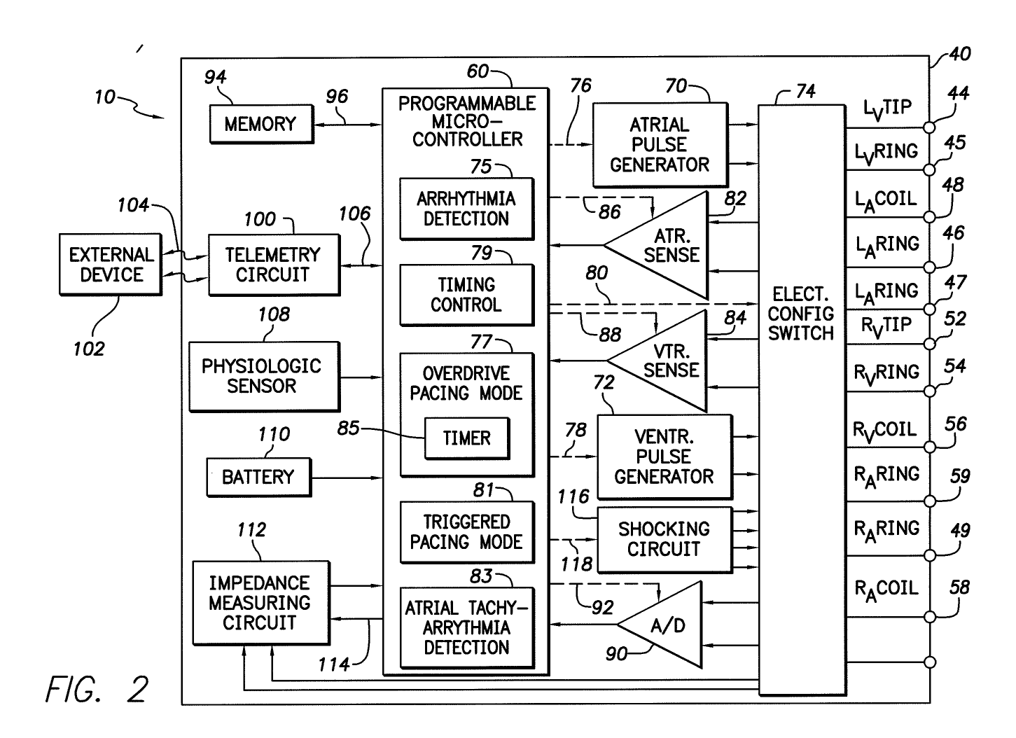

FIG. 2 illustrates a block diagram of the stimulation device 10, which is capable of treating both fast and slow arrhythmias with stimulation therapy, including cardioversion, defibrillation, overdrive pacing, standard pacing and triggered pacing stimulation. While a particular multi-chamber device is shown, this is for illustration purposes only. It is understood that the appropriate circuitry could be duplicated, eliminated or disabled in any desired combination to provide a device capable of treating the appropriate chamber(s) with cardioversion, defibrillation and pacing stimulation.

The housing 40 for the stimulation device 10, shown schematically in FIG. 2, is often referred to as the “can”, “case” or “case electrode” and may be programmably selected to act as the return electrode for some or all “unipolar” modes. The housing 40 may further be used as a return electrode alone or in combination with one or more of the coil electrodes 29, 36 and 38 of FIG. 1, for shocking purposes. The housing 40 further includes a connector (not shown) having a plurality of terminals 44, 45, 46, 47, 48, 49, 52, 54, 56, 58, and 59 (shown schematically and, for convenience, the names of the electrodes to which they are connected are shown next to the terminals). A pair of right atrial ring terminals 49 and 59 are respectively adapted for connection to first right atrial (RA) ring electrode 13 and second RA ring electrode 14 of FIG. 1. A left ventricular tip terminal 44, a left ventricular ring terminal 45, a pair of left atrial ring terminals 46 and 47, and a left atrial shocking terminal 48, are adapted for connection to the LV tip electrode 26, the LV ring electrode 25, first LA ring electrode 27 and second LA ring electrode 28, and LA coil electrode 29, respectively. A right ventricular tip terminal 52, a right ventricular ring terminal 54, a right ventricular shocking terminal 56, and an SVC shocking terminal 58, are adapted for connection to the RV tip electrode 32, RV ring electrode 34, the RV coil electrode 36, and the SVC coil electrode 38 (also know as RA coil electrode), respectively.

The stimulation device 10 includes a programmable microcontroller 60 which controls the various modes of stimulation therapy. The microcontroller 60 includes a microprocessor, or equivalent control circuitry, designed specifically for controlling the delivery of stimulation therapy and may further include RAM or ROM memory, logic and timing circuitry, state machine circuitry, and I/O circuitry. The microcontroller 60 includes the ability to process or monitor input signals (data) as controlled by a program code stored in memory. Any suitable microcontroller 60 may be used. The microcontroller 60 includes an arrhythmia detection module 75 that analyzes sensed signals and determines when an arrhythmia is occurring. The microcontroller 60 also includes an overdrive pacing mode control module 77 that may overdrive the ventricles, such as upon detection of tachycardia and/or tachyarrhythmia. A triggered pacing mode control module 81 is also provided within the microcontroller 60 for operation in conjunction with the overdrive pacing mode control module 77. The overdrive pacing mode control module 77 controls delivery of overdrive pacing pulses to a first chamber (e.g. LV or RV) in accordance with an overdrive pacing mode. The triggered pacing mode control module 81 controls delivery of a triggered pacing pulse in accordance with a triggered pacing mode. The overdrive and the triggered pacing mode control modules 77 and 81 deliver overdrive and triggered pacing pulses to a common chamber of the heart in an overlapping and temporally interspersed manner. The triggered pacing pulse may occur after an OD pacing pulse by an arbitrary interval which is independent of, and unrelated to, the timing of, and intervals between, overdrive pacing pulses.

Microcontroller 60 may include an atrial tachyarrhythmia detection control module 83 for performing a variety of tasks related to detection of atrial tachyarrhythmia and configuring the stimulation device 10. For example, the atrial tachyarrhythmia detection control module 83 may filter and process received atrial signals to detect atrial tachyarrhythmia. In response to the detection of atrial tachyarrhythmia, the atrial tachyarrhythmia detection control module 83 may select modes for sensing and modes for pacing to be used by the stimulation device 10. In general, the overdrive pacing mode control module 77, the triggered pacing mode control module 81, and the atrial tachyarrhythmia detection control module 83 typically include software, firmware, hardware and/or other computer readable medium for performing the above operations.

As shown in FIG. 2, an atrial pulse generator 70 and a ventricular pulse generator 72 generate pacing, overdrive, triggered and antitachicardia pacing (ATP) stimulation pulses for delivery by the right atrial lead 20, the right ventricular lead 30, and/or the coronary sinus lead 24 via an electrode configuration switch 74. The pulse generators 70 and 72 are controlled by the microcontroller 60 via appropriate control signals 76 and 78, respectively, to trigger or inhibit stimulation pulses. The microcontroller 60 further includes timing control circuitry 79 which is used to control the timing of such stimulation pulses (e.g., pacing rate, atrio-ventricular (AV) delay, atrial interconduction (A-A) delay, or ventricular interconduction (V-V) delay, etc.) as well as to keep track of the timing of refractory periods, PVARP intervals, noise detection windows, evoked response windows, alert intervals, marker channel timing, and the like.

The electrode configuration switch 74 includes a plurality of switches for connecting the desired electrodes to the appropriate I/O circuits. The electrode configuration switch 74, in response to a control signal 80 from the microcontroller 60, determines the polarity of the stimulation pulses (e.g., unipolar, bipolar, co-bipolar, etc.). The sensing circuits 82 and 84, receive control signals over signal lines 86 and 88 from the microcontroller 60 for purposes of controlling the gain, threshold, the polarization charge removal circuitry (not shown), and the timing of any blocking circuitry (not shown) coupled to the inputs of the sensing circuits 82 and 84.

The device 10 utilizes the atrial and ventricular sensing circuits 82 and 84 to sense cardiac signals and the arrhythmia detection module 75 to determine whether a rhythm is physiologic or pathologic. As used herein “sensing” is the receipt or noting of an electrical signal, and “detection” is the processing of these sensed signals and determining the presence of an arrhythmia. The timing intervals between sensed events (e.g., P-waves, R-waves, and depolarization signals associated with fibrillation which are sometimes referred to as “F-waves” or “Fib-waves”) are then classified by the microcontroller 60 by comparing them to a predefined rate zone limit (e.g., bradycardia, AF, normal, low rate VT, high rate VT, and fibrillation rate zones) and/or various other characteristics (e.g., sudden onset, stability, physiologic sensors, morphology, etc.) in order to determine the type of remedial therapy that is needed (e.g., biventricular pacing, bradycardia pacing, antitachicardia pacing, cardioversion shocks or defibrillation shocks, AMS pacing).

Cardiac signals are also applied to the inputs of an analog-to-digital (A/D) data acquisition system 90 which is configured to acquire intracardiac electrogram signals, convert the raw analog data into a digital signal, and store the digital signals for later processing and/or telemetric transmission to an external device 102. The data acquisition system 90 samples cardiac signals across any pair of desired electrodes.

The microcontroller 60 is further coupled to a memory 94 by a suitable data/address bus 96, wherein the programmable operating and therapy-related parameters used by the microcontroller 60 are stored and modified, as required, in order to customize the operation of the stimulation device 10 to suit the needs of a particular patient. The operating and therapy-related parameters define, for example, overdrive pacing parameters, trigger mode parameters, pacing pulse amplitude, pulse duration, electrode polarity, rate, sensitivity, automatic features, arrhythmia detection criteria, and the amplitude, wave shape and vector of each stimulating pulse to be delivered to the patient's heart 12 within each respective therapy.

The operating and therapy-related parameters may be non-invasively programmed into the memory 94 through a telemetry circuit 100 in telemetric communication with the external device 102, such as a programmer, trans-telephonic transceiver, or a diagnostic system analyzer. The telemetry circuit 100 is activated by the microcontroller 60 by a control signal 106. The telemetry circuit 100 advantageously allows intracardiac electrograms and status information relating to the operation of the device 10 (as contained in the microcontroller 60 or memory 94) to be sent to the external device 102 through an established communication link 104.

The stimulation device 10 may include a physiologic sensor 108 to adjust pacing stimulation rate according to the exercise state of the patient. The physiological sensor 108 may further be used to detect changes in cardiac output, changes in the physiological condition of the heart, or diurnal changes in activity (e.g., detecting sleep and wake states). The microcontroller 60 responds by adjusting the various pacing parameters (such as rate, AV Delay, V-V Delay, etc.) at which the atrial and ventricular pulse generators 70 and 72 generate stimulation pulses.

The battery 110 provides operating power to all of the circuits shown in FIG. 2. An impedance measuring circuit 112 monitors lead impedance during the acute and chronic phases for proper lead positioning or dislodgement; detects operable electrodes and automatically switches to an operable pair if dislodgement occurs; measures respiration or minute ventilation; measures thoracic impedance for determining shock thresholds; detects when the device has been implanted; measures stroke volume; and detects the opening of heart valves, etc.

The microcontroller 60 further controls a shocking circuit 116 by way of a control signal 118. The shocking circuit 116 generates stimulating pulses of low (up to 0.5 joules), moderate (0.5-10 joules), or high energy (11 to 40 joules), as controlled by the microcontroller 60. Stimulating pulses are applied to the patient's heart 12 through at least two shocking electrodes, which, by way of example only, may be selected from the left atrial (LA) coil electrode 29, the RV coil electrode 36 the SVC coil electrode 38 and/or the housing 40.

For patients who suffer from congestive heart failure (CHF), the device 10 may be configured as a cardiac resynchronization therapy (CRT) device that helps to synchronize the ventricular contraction pattern. Typically, these patients have a functioning AV node. Thus, electrical signals generated in the patient's atria may propagate through the AV node to the ventricles. In CRT, multisite ventricular stimulation may be triggered in response to sensed atrial events at an interval that allows the implanted system to usurp control of the normal AV nodal conduction system before intrinsic AV nodal conduction can occur.

When such a patient's heart goes into atrial tachycardia, atrial fibrillation or other pathologic atrial tachyarrhythmia, hereinafter generally referred to as atrial tachyarrhythmia, the atria may generate electrical signals at an abnormally high rate. Some of these signals may pass through the AV node to the ventricles. The introduction of these signals in the ventricles may, in turn, minimize or eliminate synchronization within the left ventricle even while the tracking mode is still in effect. When automatic mode switch (AMS) is engaged, the loss of tracking results in the native conduction system having near total control of ventricular activation. As a result, the ventricles may contract in an irregular manner, and due to the native intraventricular conduction abnormality, exacerbate low cardiac output.

When atrial tachyarrhythmia is detected, such as by the atrial tachyarrhythmia detection control module 83, the overdrive pacing mode control module 77 may be activated. During atrial tachyarrhythmia the atrium is trying to initiate conduction down the AV node at a very fast and irregular timing. The overdrive pacing mode control module 77 “overdrives” the ventricles by switching to an overdrive pacing mode that initiates a stimulating pulse in one or both of the left and right ventricles. The overdrive pacing mode has an associated mode switch base rate (MSBR) that may be a default value or may be set based on the patient. The MSBR identifies the rate of paced beats conducted by the device 10 when atrial tachyarrhythmia is detected.

In some cases, the patient's rate during atrial tachyarrhythmia may be impacted by factors such as the presence of heart failure, use of drugs such as beta blockers, poor quality AV node, and the like. Thus, some overdrive algorithms pace at a certain set and consistent rate, such as by setting the MSBR default setting to a predetermined rate, such as 60 beats per minute (bpm). In other overdrive algorithms, the MSBR may be dynamic, such as based on a patient's historical data and/or their intrinsic break through rate during atrial tachyarrhythmia, an atrial intrinsic rate, which may be higher, such as 80-90 bpm. In each case, the R-R interval, or length of time between paced beats, may be uniform. Therefore, not all of the beats will be paced beats, as some intrinsic beats during AF have intrinsic intervals that are shorter than the programmed R-R interval. Only when the intrinsic interval is longer than the programmed R-R interval will a paced beat occur.

It is desirable that a high percentage of the beats during atrial tachyarrhythmia be paced beats as CRT is only achieved when pacing the ventricle(s). Although setting the MSBR to a very high rate would achieve the goal of pacing a high percentage of the beats, this maybe undesirable. When selecting a type of overdrive algorithm and associated parameters such as MSBR, it is desirable to balance factors such as increasing the percentage of ventricular pacing and improving the regularity of the ventricular rhythm, while also maintaining an overall net average ventricular rate that is not too high. Overdrive algorithms may successfully achieve these factors at a rate of 80 to 90 percent pacing. For example, an overdrive algorithm may be dynamic such that the rate of pacing is varied to achieve 90 percent paced beats.

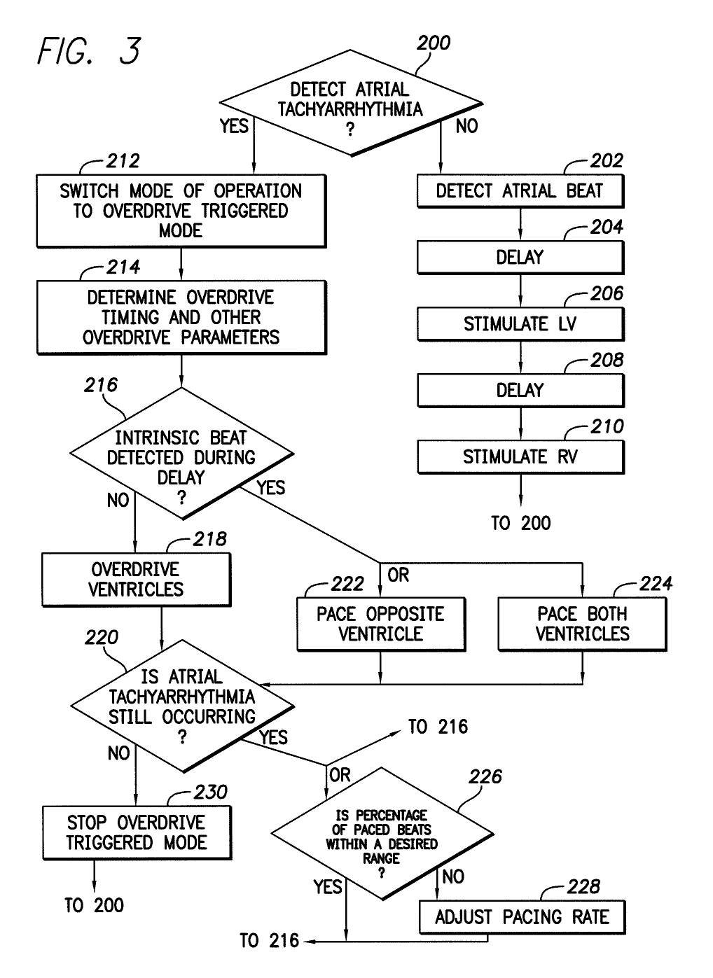

FIG. 3 illustrates a flowchart implementing a method for providing coordinated ventricular overdrive pacing and triggered pacing using the stimulation device 10 to counteract the effects of paroxysmal atrial tachyarrhythmia, such as atrial fibrillation, atrial flutter or atrial tachycardia. Overdrive pacing in the ventricles increases the regularity in the ventricular rate and increases the percentage of ventricular paced events. By also providing triggered pacing, the hemodynamics of intrinsic beats are improved. It should be understood that the overdrive pacing and triggered pacing modes may also be used upon detection of tachycardia in any other chamber or combination of chambers in the heart.

At 200, the stimulation device 10 may monitor signals from the patient to determine whether the patient's heart is experiencing some form of atrial tachyarrhythmia, resulting in the atrium potentially initiating conduction through the AV node at an undesirably fast and/or irregular rate. For example, in some embodiments, the stimulation device 10 monitors signals sensed by one or more atrial electrodes. As depicted in FIG. 1, the stimulation device 10 may monitor right atrium tip-to-ring signals (via electrodes 22 and 23), right atrium tip-to-can signals (via electrode 22 and housing 40), left atrium ring-to-can signals (via one or more of LA ring electrodes 27 and 28 and housing 40) or some combination of these signals. It should be appreciated that other methods may be used to sense signals that indicate atrial tachyarrhythmia. In another embodiment, the stimulation device 10 may seek to determine if tachycardia is occurring in any chamber of the heart, including, but not limited to, the left and/or right atrium. Further, the stimulation device 10 may seek to identify irregularities in ventricular activity based on intrinsic activity in the ventricles, without regard for activity in the atria.

The stimulation device 10 continually processes the sensed signals to determine whether the heart is currently experiencing atrial tachyarrhythmia. Typically, the stimulation device 10 periodically samples and processes the sensed signals. By way of example, the sensed signals may be amplified, filtered and sampled by the atrial sensing circuit 82. The microcontroller 60 (e.g., arrhythmia detection module 75 and/or atrial tachyarrhythmia detection control module 83) processes the signals to detect atrial tachyarrhythmia or other tachycardia. For example, the microcontroller 60 may analyze timing intervals between sensed events and classify the sensed signals according to defined limits. The microcontroller 60 also may use other techniques to detect atrial tachyarrhythmia from sensed signals.

If atrial tachyarrhythmia is not detected at 200, the stimulation device 10 may continue to provide a normal mode of pacing. The following is an exemplary pacing mode. It should be understood that other pacing modes may be used as the normal mode of pacing for a patient. At 202, the stimulation device 10 may monitor atrial signals to detect an atrial beat. Then, the stimulation device 10 delays at 204 for a defined period of time to enable the atria to assist in the filling of the ventricles. This delay period may be defined to be shorter than the time between the patient's normal atrial contraction and ventricular activation under given circumstances. The stimulation device 10 then stimulates the left ventricle at 206, delays for an appropriate period of time at 208, then stimulates the right ventricle at 210. Alternatively, the delay at 208 may be eliminated, causing the right and left ventricles to be stimulated simultaneously or nearly simultaneously. This process is repeated for every beat cycle wherein atrial tachyarrhythmia is not detected.

If atrial tachyarrhythmia is detected at 200, the microcontroller 60 may switch or change the mode of operation of the stimulation device 10 at 212 to an overdrive triggered mode, activating the overdrive pacing mode control module 77 and the triggered pacing mode control module 81. In some embodiments, the microcontroller 60 may transfer some or all processing to one or more application routines that handle processing for atrial tachyarrhythmia, such as the atrial tachyarrhythmia detection control module 83, overdrive pacing mode control module 77, and triggered pacing mode control module 81. The transfer of processing may, for example, be accomplished by generating a signal (e.g., an interrupt signal) that indicates atrial tachyarrhythmia has been detected. In this case, the interrupt may cause immediate execution of the application routines for handling atrial tachyarrhythmia. Alternatively, the microcontroller 60 may set a status indication (e.g., a flag in the memory 94) that indicates atrial tachyarrhythmia has been detected. In this case, the indication may be read by, for example, the one or more pacing routines that generate the pacing signals for each heart beat. In some embodiments, at the beginning of each beat the pacing routine(s) may check the indication to determine which mode of operation applies to the current beat.

At 214, the overdrive pacing mode control module 77 and/or microcontroller 60 may determine the pacing rate (timing) and possibly other overdrive parameters associated with the overdrive mode. For example, as discussed previously the pacing associated with the overdrive pacing mode may be preset to within a range of number of beats per minute or may be a percentage of a detected rate associated with the patient, which may be referred to as an intrinsic breakthrough rate or an atrial intrinsic rate.

As previously discussed, overdrive algorithms may overdrive one or both of the ventricles based on cardiac cycle timing, typically pacing 80 to 99 percent of the total beats. The remaining 1-20 percent of beats are intrinsic beats and thus are not CRT beats. The overdrive algorithm senses and paces in the ventricles, but pacing is inhibited by the intrinsic electrical activity of the ventricle(s). At 216, the overdrive pacing mode control module 77 monitors for an intrinsic beat, while delaying a delay period that is based on the pacing rate. An overdrive timer 85 may be used to determine one or both of start and end times of the delay period.

If no intrinsic beat is detected during the delay at 216, then at 218 the overdrive pacing mode control module 77 overdrives the ventricle(s) by delivering a series of overdrive pacing pulses in at least one of the left and right ventricles, at a designed overdrive pacing rate. The overdrive pacing rate may be determined based on the atrial intrinsic rate, a programmed rate, a rate derived from the physiology sensor and the like. At 220, the atrial tachyarrhythmia detection control module 83 determines if atrial tachyarrhythmia is still occurring, such as by sensing through, for example, right atrial lead 20. If the atrium has returned to a normal rate, the overdrive triggered mode is stopped at 230 and the device 10 is set to a preprogrammed mode. The device 10 then returns to monitor for atrial tachyarrhythmia at 200.

If atrial tachyarrhythmia is still occurring at 220, the method may return to 216, where it monitors during the next delay for an intrinsic beat. Optionally, the overdrive pacing mode control module 77 may monitor the percentage of beats being paced by the overdrive algorithm. At 226, the overdrive pacing mode control module 77 may compare the percentage of paced beats to a predetermined desired percentage or range of percentages. As discussed previously, it may be desirable to maintain a high percentage of paced beats, such as 80 or 90 percent paced beats. If the percentage of paced beats is too low, such as 50 or 60 percent of the total beats, or too high, such as 100 percent of the total beats, then at 228, the overdrive pacing model module 77 may adjust the pacing rate to either increase or decrease the pacing rate, respectively. The parameters of the overdrive timer 85 may be also adjusted accordingly. Optionally, the overdrive pacing model module 77 may adjust other algorithm parameters that are used to control the overdrive rate.

Returning to 216, if an intrinsic beat is detected during the delay period, the overdrive algorithm would not overdrive the ventricles. In other words, if operating alone, the overdrive algorithm would allow the intrinsic beat to occur, which is a poor stroke volume beat and not a CRT beat. For example, an intrinsic beat (e.g. not associated with a paced beat) may be detected by one of the coronary sinus lead 24 and right ventricular lead 30 in the left or right ventricle, respectively. By way of example, in a patient with LBBB, intrinsic activation may be sensed in the right ventricle via the right ventricular lead 30.

In one embodiment, a triggered pacing pulse is delivered through the stimulating electrode into the other ventricle or chamber. In other words, when one of the coronary sinus lead 24 and right ventricular lead 30 senses a naturally (intrinsically) occurring activation at 216, then at 222, a triggered pacing pulse is induced into the other ventricle. Continuing the example above, if the right ventricular lead 30 senses the intrinsic activation, the device 10 would then pace the left ventricle through the coronary sinus lead 24 by stimulating one or more electrodes, such as the LV ring electrode 25 and LV tip electrode 26. This causes a triggered pacing pulse to be interspersed in time with the overdrive pacing pulses. Thus, the triggered pacing pulse may occur between successive overdrive pacing pulses. The triggered pacing pulse will occur asynchronous with respect to the overdrive pacing pulses and asynchronous with respect to the pacing rate of the series of pulses forming the overdrive (OD) pacing pulses. Thus, overdrive pacing pulses may be separated by an even and/or programmed OD pulse to OD pulse interval. The triggered pacing pulse may occur after an OD pacing pulse by an arbitrary interval which is independent of, and unrelated to, the timing of, and intervals between, overdrive pacing pulses.

In an alternate embodiment, the triggered pacing mode may pace both ventricles upon detection of an intrinsic event in either ventricle. Therefore, when an intrinsic beat is detected by either of the coronary sinus lead 24 or right ventricular lead 30, then at 224, the triggered pacing mode control module 81 induces a pacing pulse into both the left and right ventricles. After the triggered pacing pulse(s) have been induced into one or both ventricle, then at 220, the device 10 again determines if atrial tachyarrhythmia is occurring.

Optionally, first and second leads having at least first and second electrodes may be located proximate to the left and right ventricles, respectively. The control module may cause delivery of the overdrive pacing pulses in both of the left and right ventricles and cause delivery of the triggered pacing pulse in only one of the left and right ventricles following detection of an intrinsic event in the other of the left and right ventricles.

By providing the triggered pacing mode, patients are relieved of the solely intrinsic activation of the ventricles for beats that are not paced by the overdrive algorithm. Quality improves as the triggered pacing mode increases the stroke volume of the beat.

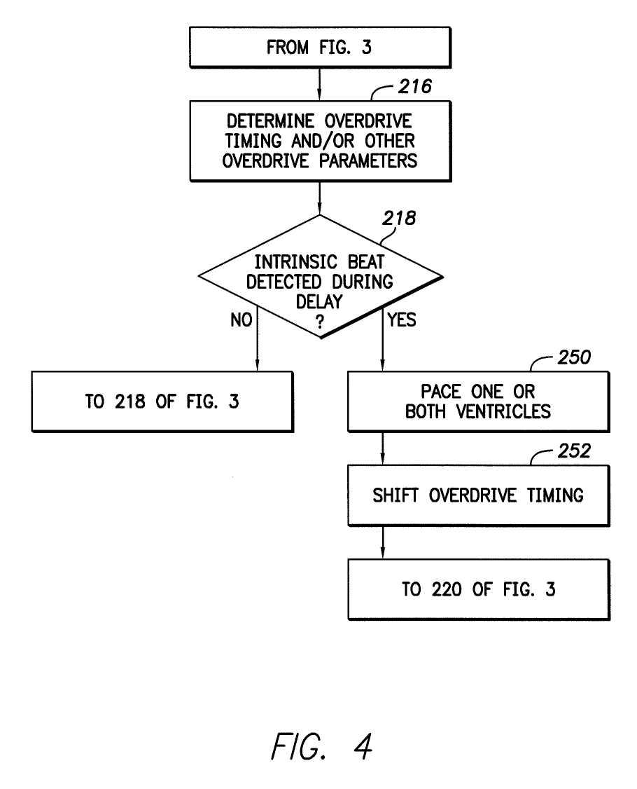

FIG. 4 illustrates a method for resynchronizing the overdrive timing based on a trigger paced event. Item numbers in common with FIG. 3 may not be discussed or will be minimally discussed with respect to FIG. 4. As in FIG. 3, when atrial tachyarrhythmia is detected at 200, the overdrive timing, such as MSBR, is determined at 214. At 216, the overdrive timer 85 may be set to monitor the delay period. When an intrinsic beat is detected during the delay period at 216, one or both of the ventricles is paced at 250, as was previously discussed. At 252 the overdrive pacing mode control module 77 may reset the start time or shift the time phase of the overdrive timer 85 relative to the intrinsic beat that lead to the triggered pacing pulse. The overdrive timer 85 may also be shifted relative to the triggered pacing pulse. Therefore, each time a triggered pacing event occurs, the delay is reset or restarted, such as at one second for 60 bpm. By shifting or resetting the overdrive timer 85, the next overdrive pacing pulse would occur one second later for 60 bpm, if no other intrinsic event occurred during the one second time period.

For example, if an intrinsic event is detected when the overdrive timer 85 is half-way through the delay period, the overdrive timer 85 would be resynchronized based on the intrinsic event such that the next paced event would occur at the desired time interval associated with MSBR, in the example above, 1 second after the paced event. Without the resynchronizing at 252, the next paced event may occur at less than the desired time interval. For example, without resetting the overdrive timer 85 the next paced event may occur at one-half the desired time interval associated with the MSBR, effectively doubling the MSBR based on those two events.

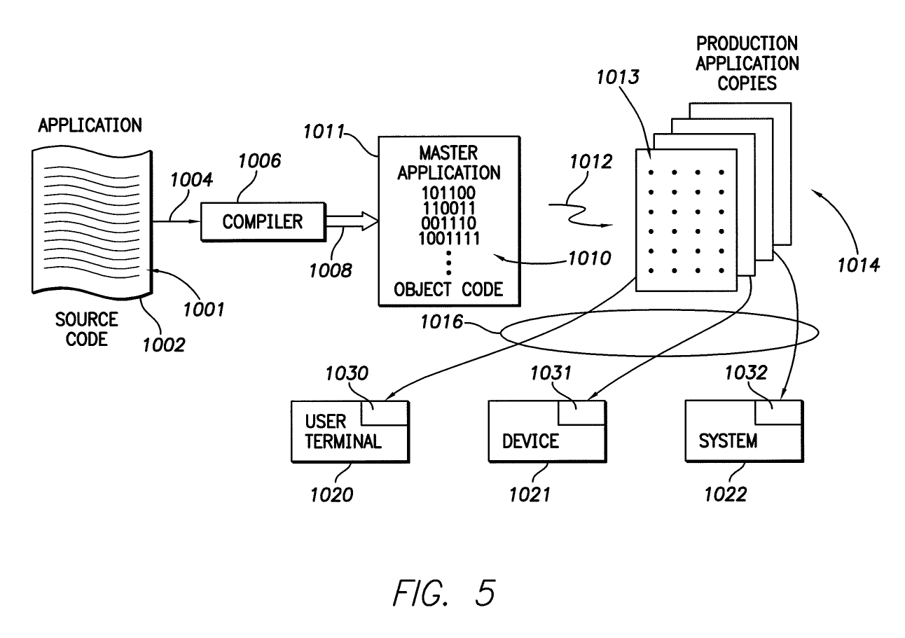

FIG. 5 illustrates a block diagram of exemplary manners in which embodiments of the present invention may be stored, distributed and installed on computer readable medium. In FIG. 5, the “application” represents one or more of the methods and process operations discussed above. For example, the application may represent the process carried out in connection with FIG. 5 as discussed above.

As shown in FIG. 5, the application is initially generated and stored as source code 1001 on a source computer readable medium 1002. The source code 1001 is then conveyed over path 1004 and processed by a compiler 1006 to produce object code 1010. The object code 1010 is conveyed over path 1008 and saved as one or more application masters on a master computer readable medium 1011. The object code 1010 is then copied numerous times, as denoted by path 1012, to produce production application copies 1013 that are saved on separate production computer readable medium 1014. The production computer readable medium 1014 is then conveyed, as denoted by path 1016, to various systems, devices, terminals and the like. In the example of FIG. 5, a user terminal 1020, a device 1021 and a system 1022 are shown as examples of hardware components, on which the production computer readable medium 1014 are installed as applications (as denoted by 1030-1032).

The source code may be written as scripts, or in any high-level or low-level language. Examples of the source, master, and production computer readable medium 1002, 1011 and 1014 include, but are not limited to, CDROM, RAM, ROM, Flash memory, RAID drives, memory on a computer system and the like. Examples of the paths 1004, 1008, 1012, and 1016 include, but are not limited to, network paths, the internet, Bluetooth, GSM, infrared wireless LANs, HIPERLAN, 3G, satellite, and the like. The paths 1004, 1008, 1012, and 1016 may also represent public or private carrier services that transport one or more physical copies of the source, master, or production computer readable medium 1002, 1011 or 1014 between two geographic locations. The paths 1004, 1008, 1012 and 1016 may represent threads carried out by one or more processors in parallel. For example, one computer may hold the source code 1001, compiler 1006 and object code 1010. Multiple computers may operate in parallel to product the production application copies 1013. The paths 1004, 1008, 1012, and 1016 may be intra-state, inter-state, intra-country, inter-country, intra-continental, intercontinental and the like.

The operations noted in FIG. 5 may be performed in a widely distributed manner world-wide with only a portion thereof being performed in the United States. For example, the application source code 1001 may be written in the United States and saved on a source computer readable medium 1002 in the United States, but transported to another country (corresponding to path 1004) before compiling, copying and installation. Alternatively, the application source code 1001 may be written in or outside of the United States, compiled at a compiler 1006 located in the United States and saved on a master computer readable medium 1011 in the United States, but the object code 1010 transported to another country (corresponding to path 1012) before copying and installation. Alternatively, the application source code 1001 and object code 1010 may be produced in or outside of the United States, but product production application copies 1013 produced in or conveyed to the United States (e.g. as part of a staging operation) before the production application copies 1013 are installed on user terminals 1020, devices 1021, and/or systems 1022 located in or outside the United States as applications 1030-1032.

As used throughout the specification and claims, the phrases “computer readable medium” and “instructions configured to” shall refer to any one or all of i) the source computer readable medium 1002 and source code 1001, ii) the master computer readable medium and object code 1010, iii) the production computer readable medium 1014 and production application copies 1013 and/or iv) the applications 1030-1032 saved in memory in the terminal 1020, device 1021 and system 1022.

It is to be understood that the above description is intended to be illustrative, and not restrictive. For example, the above-described embodiments (and/or aspects thereof may be used in combination with each other. In addition, many modifications may be made to adapt a particular situation or material to the teachings of the invention without departing from its scope. While the dimensions, types of materials and coatings described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. § 112, sixth paragraph, unless and until such claim limitations expressly use the phrase “means for” followed by a statement of function void of further structure.

What is claimed is:

1. An implantable system for providing coordinated ventricular overdrive and triggered pacing, comprising:
at least one lead for sensing signals from a heart to obtain sensed signals representative of tachycardia occurring in at least one chamber of the heart, the at least one lead including at least one electrode; and
a control module detecting tachycardia in at least one chamber of the heart and based thereon, initiating an overdrive pacing mode and a triggered pacing mode, the control module controlling delivery of overdrive pacing pulses through the electrode to a first chamber of the heart in accordance with the overdrive pacing mode, the control module controlling delivery of a triggered pacing pulse through the electrode to the first chamber of the heart in accordance with the triggered pacing mode, the triggered pacing pulse being temporally interspersed with the overdrive pacing pulses.

2. The system of claim 1, wherein the lead detects atrial activity and the control module detects one of atrial fibrillation and atrial tachyarrhythmia as the tachycardia.

3. The system of claim 1, wherein the lead detects an intrinsic event in a first ventricle and the control module causes delivery of the triggered pacing pulse to an opposite second ventricle immediately following detection of the intrinsic event in the first ventricle.

4. The system of claim 1, further comprising first and second leads having at least first and second electrodes located proximate left and right ventricles, respectively, the control module causing delivery of the triggered pacing pulse in the left ventricle immediately following detection of an intrinsic event in the right ventricle.

5. The system of claim 1, wherein the control module causes delivery of a series of overdrive pacing pulses in at least one of the ventricles at a desired pacing rate, the triggered pacing pulse occurring between successive overdrive pacing pulses asynchronous with respect to the pacing rate of the overdrive pacing pulses.

6. The system of claim 1, wherein the at least one lead comprises first and second leads located proximate the left and right ventricles.

7. The system of claim 1, further comprising first and second leads having at least first and second electrodes located proximate left and right ventricles, respectively, the control module causing delivery of the overdrive pacing pulses in both of the left and right ventricles and causing delivery of the triggered pacing pulse in only one of the left and right ventricles following detection of an intrinsic event in the other of the left and right ventricles.

8. A method of pacing ventricles during tachycardia, comprising:
sensing atrial tachycardia;
delivering overdrive pacing pulses to at least one of left and right ventricles at a pacing rate;
sensing an intrinsic event associated with at least one of the left and right ventricles; and
delivering a triggered pacing pulse to at least one of the left and right ventricles based on the intrinsic event.

9. The method of claim 8, wherein the pacing rate is based on at least one of a predetermined rate range, and a percentage of paced beats range.

10. The method of claim 8, wherein the intrinsic event is sensed in one of the left and right ventricles and the triggered pacing pulse is delivered to the opposite ventricle.

11. The method of claim 8, wherein the triggered pacing pulse is delivered to both of the left and right ventricles.

12. The method of claim 8, wherein the pacing rate has an associated overdrive timing defining a delay period between successive ones of the overdrive pacing pulses, the method further comprising shifting the overdrive timing to start a next delay period based on the triggered pacing pulse.

13. The method of claim 8, further comprising:
determining a percentage of overdrive paced beats with respect to the overdrive paced beats and the triggered paced beats occurring within a time period;
comparing the percentage to a desired predetermined percentage; and
adjusting the pacing rate based on the comparison.

14. An implantable stimulation system, comprising:
a first lead for sensing signals from a heart to obtain sensed signals representative of at least one of an intrinsic event and tachycardia occurring in at least one chamber of the heart, the first lead including at least a first electrode for delivery of a pulse to a first chamber of the heart;
a second lead for sensing signals from the heart to obtain sensed signals representative of an intrinsic event, the second lead including at least a second electrode for delivery of a pulse to a second chamber of the heart; and
an implantable stimulation device in communication with the first and second leads, the device comprising a control module configured to detect tachycardia in at least one chamber of the heart and based thereon, initiating an overdrive pacing mode and a triggered pacing mode, the control module being configured to deliver overdrive pacing pulses through at least one of the first and second electrodes in accordance with the overdrive pacing mode, the control module being configured to deliver triggered pacing pulses through at least one of the first and second electrodes upon detection of the intrinsic event.

15. The system of claim 14, wherein the triggered pacing pulses are temporally interspersed with the overdrive pacing pulses.

16. The system of claim 14, wherein one of the first and second leads detects the intrinsic event and the control module delivers the triggered pacing pulse through the other one of the first and second leads.

17. The system of claim 14, wherein one of the first and second leads detects the intrinsic event and the control module delivers the triggered pacing pulse through each of the first and second electrodes.

18. The system of claim 14, wherein successive overdrive pacing pulses are separated by a delay period, the control module shifting a start of the delay period based on at least one of the triggered pacing pulse and the intrinsic event.

19. The system of claim 14, wherein the overdrive pacing pulses are delivered based on a pacing rate, the control module adjusting the pacing rate to increase or decrease a percentage of paced beats range.
